(19) United States
(12) Patent Application Publication
(10) Pub. No.: US 2020/0383678 A1
(43) Pub. Date: Dec. 10, 2020

(54) SUTURE SYSTEM

(71) Applicant: Allan Yang Wu, Rancho Mirage, CA (US)

(72) Inventor: Allan Yang Wu, Rancho Mirage, CA (US)

(21) Appl. No.: 16/436,533

(22) Filed: Jun. 10, 2019

(51) Int. Cl. A61B 17/04

(52) U.S. Cl. CPC A61B 17/0401; A61B 17/06166; A61B 17/0482

(57) ABSTRACT

Provided herein are a suture system and a method for using the suture system to move and fixate tissue in a desired position. The suture system may have one or more anchors with protrusions extending from each body of the anchors. The protrusions may pierce and engage surrounding tissue when the suture system is inserted subcutaneously into tissue. The suture system may then be used to pull the engaged tissue in a desired direction and fixate the tissue in a desired location. Each anchor may have fenestrations disposed within each body of the anchors so that tissue may grow into the fenestrations to anchor the suture system within the engaged tissue even after the protrusions have dissolved.

### FIELD OF THE INVENTION

The invention relates to a suture system and, more specifically, to a self-anchoring suture system that has one or more anchors that may be fixed within tissue of a subject using, initially, protrusions of the suture system that engage the tissue and, subsequently, fenestrations of the suture system that permit tissue ingrowth.

### BACKGROUND OF THE INVENTION

Barbed suture materials have been used to provide initial suspension and temporary fixation of tissues for various reconstructive and cosmetic applications (e.g., plastic surgery procedures, such as face lifts). Conventional barbed sutures, however, do not provide long-term durability of tissue lifting and fixation since the barbs on the suture eventually dissolve and, consequently, the tissue surrounding the suture slides over any remaining suture material, resulting in undesired slackening of the tissue. Various solutions, such as altering the material or the quantity of the barbs themselves, have achieved only minimal improvements in durability and longevity of current cosmetic lifting techniques. It would therefore be desirable to provide improved sutures and corresponding methods for lifting and increasing fixation longevity of tissue in reconstructive and cosmetic applications.

### SUMMARY OF THE INVENTION

The above challenges are solved by a suture system that includes a suture thread with a first end connected to a first anchor. The first anchor has a body with a plurality of fenestrations sized to permit tissue ingrowth, and a plurality of protrusions extending outward from the body that are configured to anchor the suture system to a subject.

In some embodiments, the suture system is sized to pass through a carrying needle or cannula. In one or more further embodiments, a diameter of the suture thread is within a range of 0.01 mm to 5 mm, and a width of the body of the anchor is within a range of 0.01 mm to 3 cm and a thickness of the body of the anchor is within the range of 0.01 to 1 cm.

In further embodiments, the body is formed from a biodegradable polymer, a non-absorbable material, or combinations thereof. Exemplary biodegradable polymers include polydioxanone (PDS/PDO), poly lactic acid (PLA) or poly(lactic-co-glycolic- acid) (PGLA), polyglactin, polygylcolic acid, polycaprolactone, poliglycaprone 25, collagen (chromic), and combinations thereof. Examples of non-absorbable materials include a polymer, a metal alloy, a metal, a natural fiber, silk, or combinations thereof.

In further embodiments, the plurality of fenestrations includes at least three fenestration. The fenestrations are at least partially filled with a compound that enhances or accelerates tissue ingrowth, wound healing, or fibrosis. The protrusions are configured to biodegrade after substantial ingrowth of tissue, thereby maintaining the suture system in an anchored state after biodegradation of the protrusions.

In one or more embodiments, the protrusions are barbs or cones. In one or more embodiments, at least some of the protrusions extend in different directions and at least some of the protrusions have different lengths or thicknesses.

In one or more embodiments, the suture thread is formed from a biodegradable polymer.

In one or more embodiments, the suture system may also include a second anchor at an opposing end of the thread. The second anchor has a second body with a plurality of fenestrations sized to permit tissue ingrowth, and a plurality of protrusions extending outward from the second body that are configured to anchor the suture system to the subject.

In further embodiments, the first and second anchors are arranged in opposite orientations so that the first and second anchors have mirror symmetry along a plane that is orthogonal to a longitudinal axis of the suture system. In one or more embodiments, the first and second anchors are arranged in a same orientation so that the first and second anchors lack mirror symmetry along a plane that is orthogonal to a longitudinal axis of the suture system.

In other embodiments, the suture system includes a second anchor between ends of the thread. The second anchor has a second body with a plurality of fenestrations sized to permit tissue ingrowth, and a plurality of protrusions extending outward from the second body that are configured to anchor the suture system to the subject. In one or more embodiments, the first and second anchors are arranged in a same orientation so that the first and second anchors lack mirror symmetry along a plane that is orthogonal to a longitudinal axis of the suture system.

In one or more embodiments, the suture system includes a straight or curved penetrating needle.

The above challenges are also solved in a related method by securing one of the exemplary suture systems to tissue. An exemplary method includes: providing the suture system; threading the thread through the tissue so that the first anchor catches the tissue and positions at least some the plurality of fenestrations at or near an outer surface of the tissue; continuing to thread the thread through the tissue; and tying off the thread. In further embodiments, the process also includes, prior to the step of threading the thread, at least partially filling the plurality of fenestrations with a compound that enhances or accelerates tissue ingrowth, wound healing or fibrosis.

### BRIEF DESCRIPTION OF THE DRAWINGS

FIG. 1 is a pictorial diagram showing a side view of an exemplary embodiment of a suture system in accordance with one or more embodiments of the present disclosure.

FIG. 2A is a pictorial diagram showing a side view of an exemplary embodiment of the suture system in an anchored state within tissue using protrusions of an anchor in accordance with one or more embodiments of the present disclosure.

FIG. 2B is a pictorial diagram showing a side view of an exemplary embodiment of the suture system in the anchored state using fenestrations of the anchor in accordance with one or more embodiments of the present disclosure.

FIG. 3 is a pictorial diagram showing a perspective view of an exemplary embodiment of the suture system disposed within tissue in accordance with one or more embodiments of the present disclosure.

Embodiments of the present disclosure and their advantages are best understood by referring to the detailed description that follows. It should be appreciated that like reference numerals are used to identify like elements illustrated in one or more of the figures.

### DETAILED DESCRIPTION OF THE INVENTION

In accordance with various embodiments herein, a suture system is provided that may be anchored to a subject using one or more anchors of the suture system. More specifically, the suture system may be subcutaneously inserted and fixed within organic tissue to move (e.g., lift and/or pull) tissue a desired amount. For example, the suture system may be used in a cosmetic surgery to lift and tighten skin of a patient's face without being invasive and while providing long-lasting results.

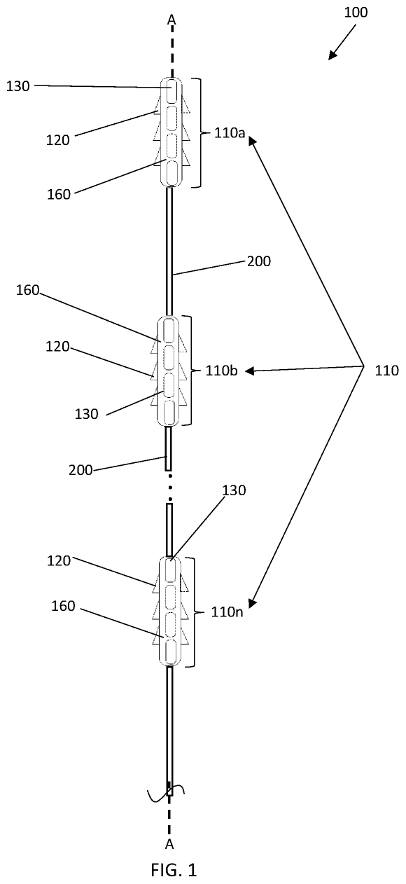

Referring now to the drawings, wherein the showings are for purposes of illustrating embodiments of the present invention only and not for the purposes of limiting the same, FIG. 1 provides an exemplary illustration of a suture system 100 in accordance with one or more embodiments of the present disclosure. Suture system 100 may include one or more anchors 110 (e.g., a first anchor 110a, a second anchor 110b, and up to an nth anchor 110n) attached to a thread 200.

In one or more embodiments, anchor 110 has a body 160 with protrusions 120 extending outward from body 160. Body 160 has one or more fenestrations 130 (e.g., four fenestrations), which are sized to permit tissue ingrowth therein. As shown in FIG. 1, anchors 110a-n may be distributed along thread 200. In other embodiments, anchors 110 may each be attached to opposing ends of thread 200. For example, suture system 100 may have two anchors 110 in which first anchor 110a may be attached to a first end of thread 200 and second anchor 110b may be attached to a second end of thread 200. Furthermore, anchors 110a,b of suture system 100 may be arranged in opposite orientations so that first anchor 110a and second anchor 110b have mirror symmetry along a plane that is orthogonal to a longitudinal axis A of suture system 100. In another example, suture system 100 may have two anchors 110, first anchor 110a and second anchor 110b, and anchors 110a,b may be arranged in a same orientation so that first anchor 110a and second anchor 110b lack mirror symmetry along a plane that is orthogonal to longitudinal axis A of suture system 100 (as shown in FIG. 1). As understood by one skilled in the art, the number of anchors 110 attached to the thread 200 and the orientations of each of the anchors 110 relative to each other may vary while still remaining within the scope of the invention.

In one or more embodiments, the orientation of each anchor 110 may be determined by the direction protrusions 120 are prominently directed. For example, protrusions 120 may be directed such that suture system 100 may traverse easily through skin in one direction, but resist traversing through the tissue in an opposing direction due to the points of protrusions 120 digging into surrounding tissue 400. For example, a user (e.g., a cosmetic surgeon) may insert suture system 100 into, for example, a jowl of a patient using a cannula. Once the leading anchor (e.g., first anchor 110a) is in a desired location in the subcutaneous layer, the cannula may be removed to expose the remainder of anchors 110b-n along thread 200. When the cannula is removed, protrusions 120 of anchors 110b-n are exposed and may engage the surrounding tissue. By providing at least two, three, or four anchors 110, the tissue 400 can be even more securely engaged, which may be preferred in some surgical methods. After insertion, the user may pull the end of thread 200, which is still exposed and extending externally from the patient's skin. The user may use various techniques to engage anchors 110 with the tissue. For example, the user may pull the exposed end of thread 200 taut to move the patient's skin a desired amount. The user may then press the epidermis to allow protrusions 120 to further dig into the patient's skin and thus maintain the pulled position of the skin so that the user no longer needs to pull or hold the exposed end of thread 200. The user may then cut the exposed end of thread 200 so that thread 200 no longer protrudes from the patient's skin, or the user may pull thread 200 and anchor the end of thread 200 using various known techniques and/or by using an anchor 110 (oriented in an opposing direction to anchor 110a) at the end of thread 200. After tissue ingrowth occurs within fenestrations 130 of anchors 110a-n, protrusions 120 and/or thread 200 may dissolve and the position of the patient's skin is maintained by anchors 110a-n.

In one or more embodiments, body 160 of anchor 110 has at least one fenestration 130 disposed therein. For example, the body 160 can have one fenestration 130, two fenestrations 130, three fenestrations 130, four fenestrations 130, five fenestrations 130, six fenestrations 130, seven fenestrations 130, eight fenestrations 130, nine fenestrations 130, ten fenestrations 130, eleven fenestrations 130, twelve fenestrations 130, thirteen fenestrations 130, fourteen fenestrations130, fifteen fenestrations 130 or more. Fenestrations 130 are sized to permit tissue ingrowth. More specifically, tissue of a subject that surrounds suture system 100 encapsulates suture system 100 and grows within and/or through one or more of the fenestrations 130. As a result of the tissue ingrowth, the position of anchor 110 within the tissue is fixed (e.g., suture system 100 is in an anchored state), and thus undesired movement of the tissue about suture system 100 is prevented, even if protrusions 120 of anchor 110 dissolve (as described further herein).

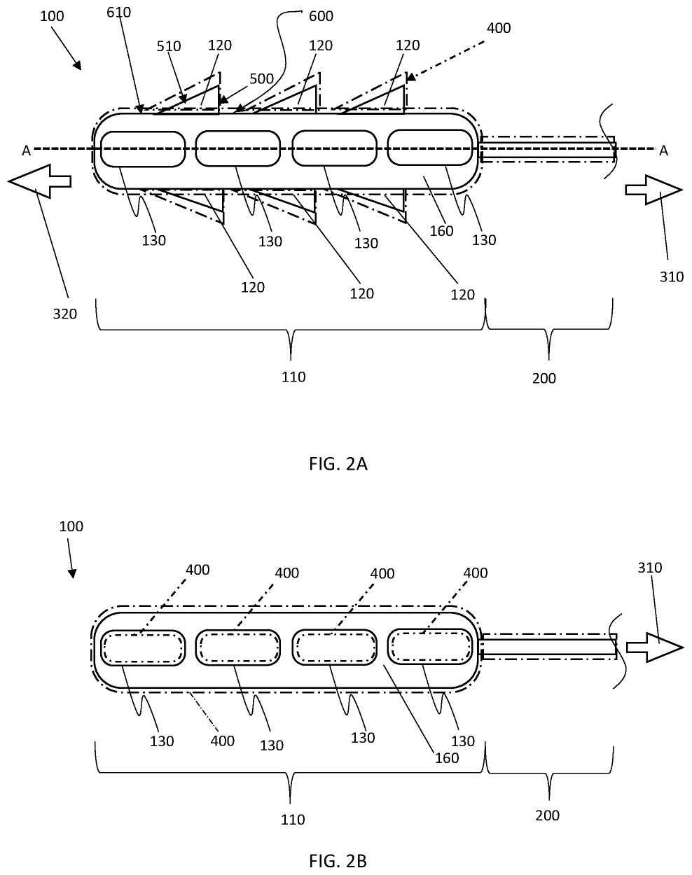

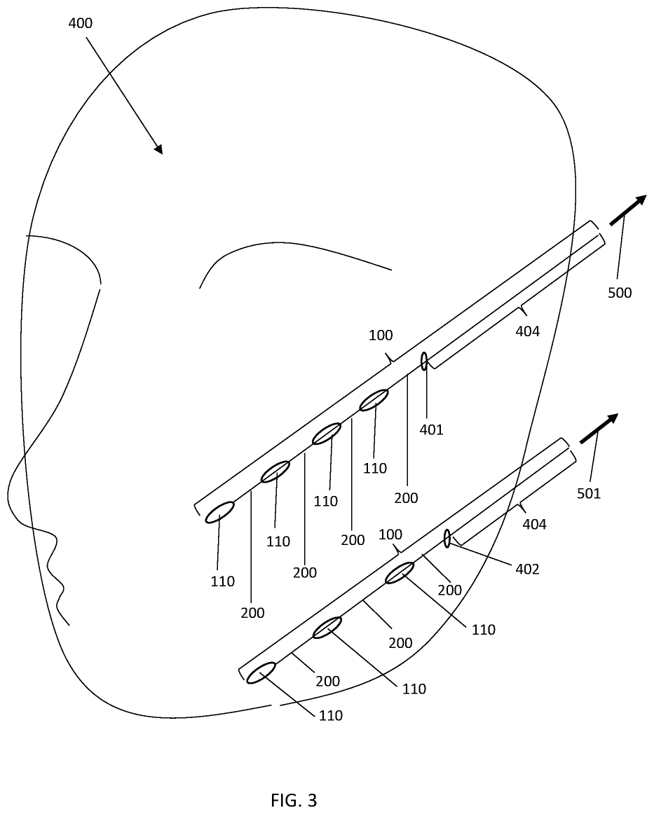

FIG. 2A illustrates a side view of an exemplary embodiment of suture system 100 in an anchored state within tissue 400 (see FIG. 3) using protrusions 120 of anchor 110 in accordance with one or more embodiments of the present disclosure. Protrusions 120 of anchor 110 extend from body 160 and prevent outward movement of anchor 110 from within tissue 400 once anchor 110 has been inserted subcutaneously into tissue 400. In one or more embodiments, protrusions 120 may have a pointed end and may be formed from a rigid material that allows protrusions 120 to pierce tissue 400. In one or more embodiments, protrusions 120 may be unidirectional angled barbs that extend from body 160. Protrusions 120 oriented unidirectionally permit anchor 110 to easily traverse subcutaneously through tissue 400 in a direction, such as a direction indicated by arrow 320, but create resistance (e.g., snag or dig into tissue 400 to engage tissue 400) and prevent movement of anchor 110 in an opposing direction, such as in a direction indicated by arrow 310. In other embodiments, protrusions 120 may extend in different directions relative to each other. Thus, protrusions 120 may provide resistance against various forces pulling on anchor 110 (e.g., a force pulling in the direction indicated by arrow 310) once anchor 110 is inserted into a desired location within tissue 400. Furthermore, the resistance provided by protrusions 120 of anchor 110 allows engaged tissue 400 to be shifted (e.g., pulled and/or lifted) a desired amount by a user since anchor 110 cannot not traverse within the tissue once protrusions 120 are engaged with surrounding tissue 400. For example, if thread 200 is pulled by a user or by an opposing anchor, then tissue 400 is moved in the direction of the pulling force due to embedded anchor 110 providing resistance against the pulling force and creating tension along thread 200.

In one or more embodiments, a plurality of protrusions 120 extend outward from body 160 and are configured to anchor suture system 100 to a subject (e.g., a patient). In one or more embodiments, protrusions 120 are illustrated as cone-shaped protrusions in the exemplary illustrations; however, as understood by one skilled in the art, protrusions 120 may be various other shapes, sizes, and thicknesses. In one or more embodiments, protrusions 120 may be straight or curved barbs, cones, and/or other shapes with pointed ends. In one or more embodiments, protrusions 120 may be oriented unidirectionally, bidirectionally, or in varying directions relative to each other. In one or more embodiments, protrusions 120 may have different lengths or thicknesses. In other embodiments, protrusions 120 may be uniformly sized and shaped. In one or more embodiments, protrusions 120 may be positioned linearly on opposing sides of anchor 110 relative to central axis A. In other embodiments, protrusions 120 may be positioned radially, helically, randomly, and/or positioned in any other various arrangements about body 160.

In one or more embodiments, body 160 and/or protrusions 120 may each be made from absorbable and/or non-absorbable biocompatible materials. Body 160 and/or protrusions 120 may each be formed from a biodegradable material (e.g., a biodegradable polymer), a non-absorbable material, or combinations thereof. In one or more embodiments, the biodegrabable polymer is polydioxanone (PDS/PDO), poly lactic acid (PLA) or poly(lactic-co-glycolic-acid) (PGLA), polyglactin, polygylcolic acid, polycaprolactone, poliglycaprone 25, collagen (chromic), or combinations thereof. In one or more embodiments, the non-absorbable material is a polymer (e.g., polypropylene, polyamide, polyester, polyetherester, polytetrafluoroethylene), a metal alloy, a metal, a natural fiber, silk, or combinations thereof. In one or more embodiments, body 160 and/or protrusions 120 may be formed using various suitable methods, such as injection molding, cutting, stamping, lasering, etc.

In one or more embodiments, protrusions 120 may be configured to biodegrade after substantial ingrowth of tissue 400 within fenestrations 130. For example, protrusions 120 may be formed from an absorbable material such that, after suture system 100 is inserted into tissue 400, protrusions 120 are configured to biodegrade within a duration of time that permits tissue ingrowth within fenestrations 130 so that fenestrations 130 maintain the anchored state of suture system 100.

As understood by one skilled in the art, though fenestrations 130 are illustrated as linearly oriented within body 160, fenestrations 130 may be arranged in various other arrangements (e.g., grid, circular, random, etc.). Furthermore, fenestrations 130 may also be various uniform or nonuniform shapes (e.g., square, circle, triangle, oblong, etc.). In one or more embodiments, fenestrations 130 extend through the entire thickness of body 160.

Thread 200 may be made from various materials depending on the desired stress-load ratio, tensile strength, retention rate, flexibility, thermal properties, elasticity, and other desired properties for the particular procedure usage. In one or more embodiments, thread 200 may be made from an absorbable material, non-absorbable material, or any combinations thereof. In one or more embodiments, thread 200 is formed from a biodegradable polymer. In one or more embodiments, thread 200 may be made from an absorbable synthetic material (e.g., monocryl, vicryl, polydioxanone (PDS)) or absorbable natural material (e.g. material made from natural fibers such as collagens). Furthermore, thread 200 may be made from a non-absorbable synthetic material (e.g., ethilon or prolene) or a non-absorbable natural material (e.g., surgical cotton, surgical silk, or surgical steel). In one or more embodiments, thread 200 may be made of one filament (i.e. a monofilament suture) or multiple filaments (i.e. a multifilament suture). Thread 200 may be made with various diameters, lengths, and grades depending on, for example, the desired tensile strength and handling ability by a user (e.g., a cosmetic surgeon). In one or more embodiments, thread 200 may have a diameter range within 0.01 mm and 5 mm. In some embodiments, the thread has a diameter of 0.5 mm. In some embodiments, the thread has a diameter of 1 mm. In some embodiments, the thread has a diameter of 1.2 mm. In some embodiments, the thread has a diameter of 1.5 mm. In some embodiments, the thread has a diameter of 2 mm. In some embodiments, the thread has a diameter of 2.5 mm. In some embodiments, the thread has a diameter of 3 mm. In some embodiments, the thread has a diameter of 3.5 mm. In some embodiments, the thread has a diameter of 4 mm. In some embodiments, the thread has a diameter of 4.5 mm. In some embodiments, the thread has a diameter of 5 mm. In preferred embodiments, both thread 200 and anchors 110 have diameters that allow suture system 100 to be inserted into tissue 400 using, for example, a cannula or a curved or straight carrying needle. In one or more embodiments, body 160 may have a width within a range of 0.01 mm to 3 cm. In one or more embodiments, body may have a thickness within a range of 0.01 cm to 1 cm.

FIG. 2B depicts an exemplary suture system 100 anchored in tissue 400 using ingrowth of tissue 400 within fenestrations 130 after projections 120 have dissolved in accordance with one or more embodiments of the disclosure. More specifically, anchor 110 may be inserted subcutaneously into tissue 400 and, after a duration of time, tissue 400 grows into fenestrations 130 of anchor 110, thus fixing the position of anchor 110 within tissue 400. Thus, suture system 100 is anchored within tissue 400 by the ingrowth of tissue 400 within fenestrations 130. The ingrowth of tissue 400 secures anchor 110 within tissue 400 and prevents anchor 110 from undesirably traversing within tissue 400 even after protrusions 120, which initially secured anchor 110 within tissue 400, have dissolved. In one or more embodiments, one or more medicaments may be placed within apertures 130 to expedite ingrowth of tissue 400 within fenestrations 130 and/or to prevent bacterial infections. Thus, in one or more embodiments, fenestrations 130 may at least partially be filled with a compound that enhances or accelerates tissue ingrowth, wound healing, and/or fibrosis.

As shown in FIG. 3, suture system 100 may be used in reconstructive or cosmetic applications in accordance with one or more embodiments of the present disclosure. For example, a midface lift procedure may be achieved using one or more suture systems 100. First, a surgeon may insert anchor 110 of suture system 100 subcutaneously into the malar area of a patient 400 (through an incision made by the surgeon, such as incisions 401 and 402) using a cannula or needle, as previously mentioned herein. Anchors 110a,b may then engage the subcutaneous layer of the skin of patient 400. Exposed end 404 of thread 200 may be pulled taut by the surgeon (as indicated by force vectors 500 and 501) until anchors 110a,b and the tissue engaged by anchors 110a,b are lifted a desired amount such that the lifted tissue in the malar area creates a more youthful appearance in the face of patient 400 by reducing nasolabial lines and restoring cheek volume. Opposing end 404 of thread 200 may be fixed subcutaneously within the skin of the patient's forehead using an opposing anchor to maintain the tension of thread 200, or end 404 of thread 200 may be cut so that the remainder of thread 200 may reside beneath the epidermis. After a duration of time, the tissue surrounding suture system 100 will grow within fenestrations 130. Thus, if protrusions 120 and/or thread 200 dissolve after a duration of time, the lift of the tissue is maintained because of the tissue ingrowth within fenestrations 130.

In one or more embodiments, a method of anchoring suture system 100 within tissue may include one or more of the following procedures described herein. Anchor 110 may be slidably disposed within tissue by anchor 110 subcutaneously traversing through the tissue in a first direction. Once anchor 110 is positioned in the desired location within the tissue, thread 200 may be pulled in a second, opposing direction, which results in protrusions 120 digging into the surrounding tissue (e.g., the sharp ends of protrusions 120 may snag and/or pierce the tissue). As a result, movement (e.g., rotation or slippage) of anchor 110 is prevented. Furthermore, the resistance provided by anchor 110 permits anchor 110 to pull on the tissue and move the tissue in a relatively parallel direction to the second direction. The skin may be pressed or rubbed to further engage protrusions 120 with surrounding tissue and prevent movement of the surrounding tissue about suture system 100. Protrusions 120 may maintain the position of the lifted skin until tissue ingrowth occurs in fenestrations 130. Once tissue has substantially grown within fenestrations 130, protrusions 120 and/or thread 200 may dissolve, and the lifted position of the tissue will still be maintained by fenestrations 130.

In an embodiment of the disclosure, suture system 100 may be inserted into a cannula (e.g., medical sleeve) so that suture system 100 may then be readily threaded and embedded into tissue using techniques and methods known to those skilled in the art. In another embodiment, suture system 100 may be threaded through a carrying needle and inserted into the tissue 400. For example, a process may include threading thread 200 through the tissue 400 so that first anchor 110a catches the tissue 400 and positions at least some the plurality of fenestrations 130 at or near an outer surface of the tissue. Another step may then include continuing to thread the thread 200 through the tissue 400. The process may also include a step of tying off thread 200. Prior to the step of threading the thread 200, a process of at least partially filling fenestrations 130 with a compound that enhances or accelerates tissue ingrowth, wound healing, or fibrosis may be done.

The disclosure is not intended to limit the present invention to the precise forms or particular fields of use disclosed. It is contemplated that various alternate embodiments and/or modifications to the present invention, whether explicitly described or implied herein, are possible in the suture system of the disclosure. For example, it is contemplated that the various embodiments set forth herein may be combined together and/or separated into additional embodiments where appropriate.

While the invention has been described in detail in connection with only a limited number of embodiments, it should be readily understood that the invention is not limited to such disclosed embodiments. Rather, the invention can be modified to incorporate any number of variations, alterations, substitutions, or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A suture system comprising a suture thread with a first end connected to a first anchor, the first anchor comprising a body with a plurality of fenestrations sized to permit tissue ingrowth; and a plurality of protrusions extending outward from the body that are configured to anchor the suture system to a subject.

2. The suture system of claim 1, wherein the suture system is sized to pass through a carrying needle or cannula.

3. The suture system of claim 1, wherein:
a diameter of the suture thread is within a range of 0.01 mm to 5 mm; and
a width of the body of the anchor is within a range of 0.01 mm to 3 cm and a thickness of the body of the anchor is within the range of 0.01 to 1 cm.

4. The suture system of claim 1, wherein the body is formed from a biodegradable polymer, a non-absorbable material, or combinations thereof.

5. The suture system of claim 4, wherein:
the biodegradable polymer is polydioxanone (PDS/PDO), poly lactic acid (PLA) or poly(lactic-co-glycolic- acid) (PGLA), polyglactin, polygylcolic acid, polycaprolactone, poliglycaprone 25, collagen (chromic), and combinations thereof, and
the non-absorbable material is a polymer, a metal alloy, a metal, a natural fiber, silk, or combinations thereof.

6. The suture system of claim 1, wherein the plurality of fenestrations comprises at least one fenestration.

7. The suture system of claim 1, wherein the fenestrations are at least partially filled with a compound that enhances or accelerates tissue ingrowth, wound healing, or fibrosis.

8. The suture system of claim 7, wherein the protrusions are configured to biodegrade after substantial ingrowth of tissue, thereby maintaining the suture system in an anchored state after biodegradation of the protrusions.

9. The suture system of claim 1, wherein the protrusions are barbs or cones.

10. The suture system of claim 1, wherein at least some of the protrusions extend in different directions.

11. The suture system of claim 1, wherein at least some of the protrusions have different lengths or thicknesses.

12. The suture system of claim 1, wherein the suture thread is formed from a biodegradable polymer.

13. The suture system of claim 1, further comprising a second anchor at an opposing end of the thread, the second anchor comprising a second body with a plurality of fenestrations sized to permit tissue ingrowth; and a plurality of protrusions extending outward from the second body that are configured to anchor the suture system to the subject.

14. The suture system of claim 13, wherein the first and second anchors are arranged in opposite orientations so that the first and second anchors have mirror symmetry along a plane that is orthogonal to a longitudinal axis of the suture system.

15. The suture system of claim 13, wherein the first and second anchors are arranged in a same orientation so that the first and second anchors lack mirror symmetry along a plane that is orthogonal to a longitudinal axis of the suture system.

16. The suture system of claim 1, further comprising a second anchor between ends of the thread, the second anchor comprising a second body with a plurality of fenestrations sized to permit tissue ingrowth; and a plurality of protrusions extending outward from the second body that are configured to anchor the suture system to the subject.

17. The suture system of claim 16, wherein the first and second anchors are arranged in a same orientation so that the first and second anchors lack mirror symmetry along a plane that is orthogonal to a longitudinal axis of the suture system.

18. The suture system of claim 1, further comprising a straight or curved penetrating needle.

19. A method of securing a suture system to tissue, the method comprising:
providing the suture system of claim 1;
threading the thread through the tissue so that the first anchor catches the tissue and positions at least some the plurality of fenestrations at or near an outer surface of the tissue;
continuing to thread the thread through the tissue; and
tying off the thread.

20. The method of claim 19, further comprising, prior to the step of threading the thread, at least partially filling the plurality of fenestrations with a compound that enhances or accelerates tissue ingrowth, wound healing, or fibrosis.
